(19) United States
(12) Patent Application Publication
(10) Pub. No.: US 2022/0354430 A1
(43) Pub. Date: Nov. 10, 2022

(54) STENT APPARATUSES AND RELATED SYSTEMS AND METHODS FOR PROTECTION AND MAPPING OF BODILY TISSUES

(71) Applicants: Michael Hobart, Edmonton (CA); Adam Kinnaird, Edmonton (CA); James Kinnaird, Edmonton (CA)

(72) Inventors: Michael Hobart, Edmonton (CA); Adam Kinnaird, Edmonton (CA); James Kinnaird, Edmonton (CA)

(21) Appl. No.: 17/738,886

(22) Filed: May 6, 2022

Related U.S. Application Data
(60) Provisional application No. 63/185,722, filed on May 7, 2021.

(51) Int. Cl. A61B 5/00; A61B 5/06; A61B 5/0536; A61B 5/0538

(52) U.S. Cl. CPC A61B 5/6862; A61B 5/063; A61B 5/0536; A61B 5/0538; A61B 2505/05; A61B 2560/0276; A61B 2562/166

(57) ABSTRACT

Stent apparatuses are provided. In some embodiments, the stent apparatus comprises: a tubular body; and at least two induction loops capable of generating an electrical current in the presence of one or more of magnetic, electrical, and radiofrequency energy. The stent apparatus may be placed in a non-target bodily tissue during surgery and used to sense the proximity of an electrosurgical instrument to prevent injury to the non-target tissue. Also provided herein are related systems and methods for protecting and/or mapping the non-target bodily tissue having the stent apparatus placed therein.

### CROSS REFERENCE TO RELATED APPLICATION

The present application claims priority to U.S. Provisional Patent Application No. 63/185,722, filed May 7, 2021, the entire contents of which are incorporated herein by reference.

### TECHNICAL FIELD

The present disclosure relates to surgical instruments. More particularly, the present disclosure relates to stents and related systems and methods for protection and mapping of bodily tissues.

### BACKGROUND

Injury to non-target tissues is a common and serious complication in surgeries. In particular, ureteral injuries are common during pelvic, gynecological, obstetrical, and colorectal surgeries. The ureter may by obscured by fat or other surrounding tissues or may resemble another tissue that is the target of the surgery. A surgical instrument, such as an electrocautery probe, may inadvertently contact the ureter resulting in damage to the tissue.

Prophylactic preoperative ureteric stent insertion may be used to assist visualization and palpation of the ureter by the surgeon. Some ureteric stents are also lighted to further improve visualization. However, previous studies have shown that these stents only improve detection of injuries once they have happened and do not prevent injuries from occurring. As a result, ureteral injuries can lead to patient morbidity and additional hospital costs, including additional surgery to repair the damage.

Other non-target tissues that may be injured during surgery include vascular tissue, intestinal tissue and the rectum.

### SUMMARY

In one aspect, there is provided a stent apparatus comprising: a tubular body to be positioned in a bodily tissue; at least one pair of induction loops in or on the tubular body, the at least one pair of induction loops comprising a first induction loop and a second induction loop, the first induction loop substantially orthogonal to the second induction loop; and wherein the at least one pair of induction loops is capable of generating an electrical current in the presence of one or more of magnetic, electrical, and radiofrequency energy.

In some embodiments, each induction loop of the at least one pair of induction loops comprises a respective coiled wire.

In some embodiments, the at least one pair of induction loops is embedded in the tubular body.

In some embodiments, each induction loop of the at least one pair of induction loops comprises conductive ink printed on the tubular body.

In some embodiments, the at least one pair of induction loops is disposed on an outer surface of the tubular body.

In some embodiments, the at least one pair of induction loops further comprises a third induction loop and a fourth induction loop, the third induction loop substantially orthogonal to the fourth induction loop.

In some embodiments, the stent apparatus further comprises at least one of a pressure sensor, a temperature sensor, and a light source.

In another aspect, there is provided a system for sensing a surgical instrument in a bodily tissue, comprising: a stent apparatus to be positioned in the bodily tissue, the stent apparatus comprising at least one pair of induction loops that generate a signal in the presence of the surgical instrument; and a control system operatively connected to the stent apparatus, the control system comprising a sensor operable to receive the signal from the at least one pair of induction loops.

In some embodiments, the signal is an electrical current and the sensor comprises a galvanometer or an ammeter.

In some embodiments, the control system further comprises a user interface that generates a proximity indicator in response to the signal from the stent apparatus.

In some embodiments, the proximity indicator is an audible or visual output signal.

In some embodiments, the control system is operatively connected to a generator that supplies power to the surgical instrument, and wherein the control system is operable to transmit a control signal to the generator to shut off power to the surgical instrument in response to the signal from the stent apparatus.

In another aspect, there is provided a method at a control system comprising a sensor, the control system operatively connected to a surgical instrument and a stent apparatus comprising at least one pair of induction loops, the method comprising: powering the surgical instrument; and sensing a signal, via the sensor, generated from the at least one pair of induction loops when the surgical instrument approaches the stent apparatus.

In some embodiments, the method further comprises shutting off power to the surgical instrument in response to the signal.

In some embodiments, power is shut off when the signal reaches a pre-determined threshold, the pre-determined threshold being based on one or more of a safe distance, temperature, time/duration, voltage and amperage of the surgical instrument.

In some embodiments, powering the surgical instrument comprises supplying a low current to the surgical instrument, the low current being atraumatic to tissue.

In some embodiments, the method further comprises generating a proximity indicator in response to the signal, the proximity indicator comprising an audible or visual output signal.

In some embodiments, the intensity of the audible or visual output signal varies in response to variations in the signal when the surgical instrument moves closer or farther away from the stent apparatus.

In some embodiments, the method further comprises processing the signal to generate an image of a non-target bodily tissue.

In some embodiments, the method further comprises sensing a decrease or loss of the signal to indicate damage to the stent apparatus.

Other aspects and features of the present disclosure will become apparent, to those ordinarily skilled in the art, upon review of the following description of the specific embodiments of the disclosure.

### BRIEF DESCRIPTION OF THE DRAWINGS

Some aspects of the disclosure will now be described in greater detail with reference to the accompanying drawings. In the drawings:

FIG. 1 is a perspective view of an example stent apparatus, according to some embodiments;

FIG. 2 is a perspective view of an example system including the apparatus of FIG. 1, according to some embodiments;

FIG. 3 is a block diagram of a control system of the system of FIG. 2, according to some embodiments;

FIG. 4 is a flowchart of a method for protecting a non-target tissue implemented using the system of FIG. 2, according to some embodiments; and

FIG. 5 is a flowchart of a method for mapping a non-target tissue implemented using the system of FIG. 2, according to some embodiments.

### DETAILED DESCRIPTION

Generally, the present disclosure provides a stent apparatus. In some embodiments, the stent apparatus comprises: a tubular body; and at least two induction loops capable of generating an electrical current in the presence of magnetic, electrical, and/or radiofrequency energy via Faraday's Law. Also provided herein is a related system including the stent apparatus and related methods for protecting and/or mapping a non-target bodily tissue.

As used herein the terms “a,” “an,” and “the” may include plural referents unless the context clearly dictates otherwise.

As used herein, the term “stent” refers to any tubular structure that may be placed in a human body.

As used herein, the term “subject” or “patient” refers to any individual including a human or another animal. The subject may be an adult or a child.

As used herein, the term “user” refers to one or more individuals using the apparatuses and systems described herein including, but not limited to, a surgeon or another medical professional.

As used herein, “tissue” refers to any structure or aggregate of cells and intercellular matter in the body of the subject, inclusive of organs and parts thereof. A “target tissue” refers to a bodily tissue of the subject that is the target of a surgical operation and “non-target tissue” refers to a bodily tissue of the subject that is not the target of the operation and/or any tissue to which it is desired to avoid damage.

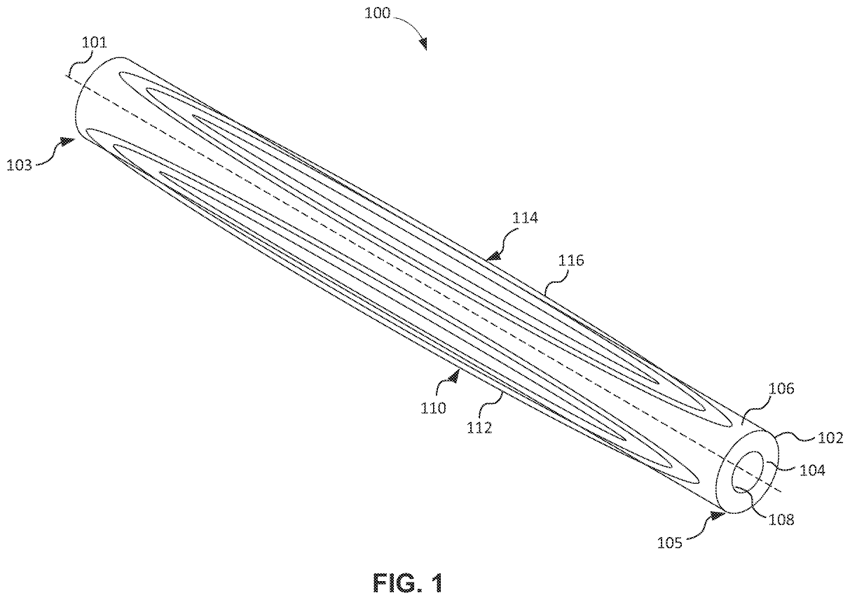

An example stent apparatus 100 will be described with reference to FIG. 1.

The apparatus 100 in this embodiment comprises an elongate tubular body 102 having a first end 103 and a second end 105 and extending along a longitudinal axis 101. The tubular body 102 may define an axial bore 104 extending through the tubular body 102 from the first end 103 to the second end 105. The tubular body 102 has an outer surface 106 and an inner surface 108, the inner surface 108 defining the axial bore 104. In this embodiment, the tubular body 102 and the axial bore 104 are each substantially cylindrical in shape. In other embodiments, each of the tubular body 102 and axial bore 104 may be any other suitable shape.

The tubular body 102 may be comprised of any suitable material, including but not limited to materials used to make conventional stents. Non-limiting examples of suitable materials include polyethylene, polyurethane, Polytetrafluoroethylene, silicone rubber, nylon, latex, thermoplastic elastomers, and other polymers.

The tubular body 102 may be configured to be positioned in a desired non-target tissue of a subject. In this embodiment, the non-target tissue is a ureter of the subject. In other embodiments, the non-target tissue is vascular tissue, intestinal tissue, rectal tissue, or any other suitable bodily tissue.

The tubular body 102 may have a suitable diameter based on the non-target tissue in which it is to be placed. In this embodiment, the tubular body 102 has a diameter of 5-8 F or about 1.6 to about 2.6 mm in diameter. The tubular body 102 may have a suitable length including but not limited to 18, 22, 24, 26, 30, and 32 cm. In other embodiments, the tubular body 102 may be any other suitable length and diameter.

The apparatus 100 may further comprise at least one pair of induction loops in or on the tubular body 102. As used herein, “induction loop” refers to a conductive path forming at least one loop or turn of a coil. In this embodiment, the apparatus 100 comprises a first induction loop 110 and a second induction loop 114. The first induction loop 110 is substantially orthogonal to the second induction loop 114 (i.e., in substantially perpendicular planes). In other embodiments, the first and second induction loops 110 and 114 may be in another suitable configuration.

In this embodiment, each of the first and second induction loops 110 and 114 comprises a respective coiled wire (i.e., a multi-turn loop). The first induction loop 110 comprises a first coiled wire 112 and the second induction loop 114 comprises a second coiled wire 116. Each coiled wire 112, 116 may comprise an electrical wire such as, for example, a wrapped insulated narrow-gauge wire. The first coiled wire 112 and the second coiled wire 116 may be interconnected to provide a substantially continuous circuit. The first coiled wire 112 and the second coiled wire 116 in this embodiment are each elongated along the longitudinal axis 101 such that they form an approximately elliptical shape. In other embodiments, each of the coiled wires 112, 116 may be coiled in an approximately circular, square, rectangular, or triangular shape or any other suitable shape.

In some embodiments, the first and second coiled wires 112 and 116 are embedded in the tubular body 102. For example, the tubular body 102 may be formed by injection moulding or 3D-printing with the coiled wires 112 and 116 integrated therein. In other embodiments, the first and second coiled wires 112 and 116 are disposed on the outer surface 106 of the tubular body 102. For example, the coiled wires 112 and 116 may be coupled to the outer surface 106 by an adhesive or any other suitable means. In yet other embodiments, the first and second coiled wires 112 and 116 are disposed within the axial bore 104, for example, by coupling the coiled wires 112, 116 to the inner surface 108.

In an alternative embodiment, the first and second induction loops 110 and 114 may comprise conductive ink. The conductive ink may be printed on the outer surface 106 of the tubular body 102. The conductive ink may be printed in the form of a pair of multi-loop coils, similar to the coiled wires 112, 116 of FIG. 1. The conductive ink may be printed to form a substantially continuous circuit.

Optionally, the apparatus 100 may comprise one or more additional pairs of induction loops (not shown). For example, the apparatus 100 may comprise a third induction loop and a fourth induction loop, the third induction loop substantially orthogonal to the fourth induction loop. The third and fourth induction loops may be similar in structure to the first and second induction loops 110 and 114 as described above.

In use, the apparatus 100 may be placed in the desired non-target tissue and the first and second induction loops 110 and 114 may generate a signal, such as an electrical current, via Faraday's Law of induction in the presence of magnetic, electrical, and/or radiofrequency energy. The apparatus 100 may thus be used to sense the proximity of an electrosurgical instrument and thereby protect and/or map the non-target tissue as described in more detail below. The presence of the induction loops 110, 114 in or on the tubular body 102 may also strengthen the stent apparatus 100 to reduce the risk of damage to the tubular body 102, while still maintaining flexibility to allow the stent apparatus 100 to be inserted into a non-target bodily tissue such as the ureter or rectum.

Optionally, the apparatus 100 further comprises one or more sensors (not shown). For example, the apparatus 100 may comprise at least one of a temperature (heat) sensor and a pressure sensor. For example, the apparatus 100 may include one or more thermistors, strain gauges, or any other equivalent or similar sensor. The temperature sensor may be used to detect a change in temperature in the area of the non-target tissue, for example, an increase in temperature due to the close proximity of an electrosurgical instrument. The pressure sensor may be used to indicate if the apparatus 100 has been crushed or damaged and thus if the non-target tissue may have a crush injury. The temperature and/or pressure sensor may be embedded in the tubular body 102 or may be coupled to the tubular body 102 by any suitable means.

Optionally, the apparatus 100 further comprises a light source (not shown). The light source may comprise one or more lights including, for example, LED (light emitting diode) lights. The light source may aid in visualization of the apparatus 100 during surgery. The light source may be embedded in the tubular body 102 or may be coupled to the tubular body 102 by any suitable means.

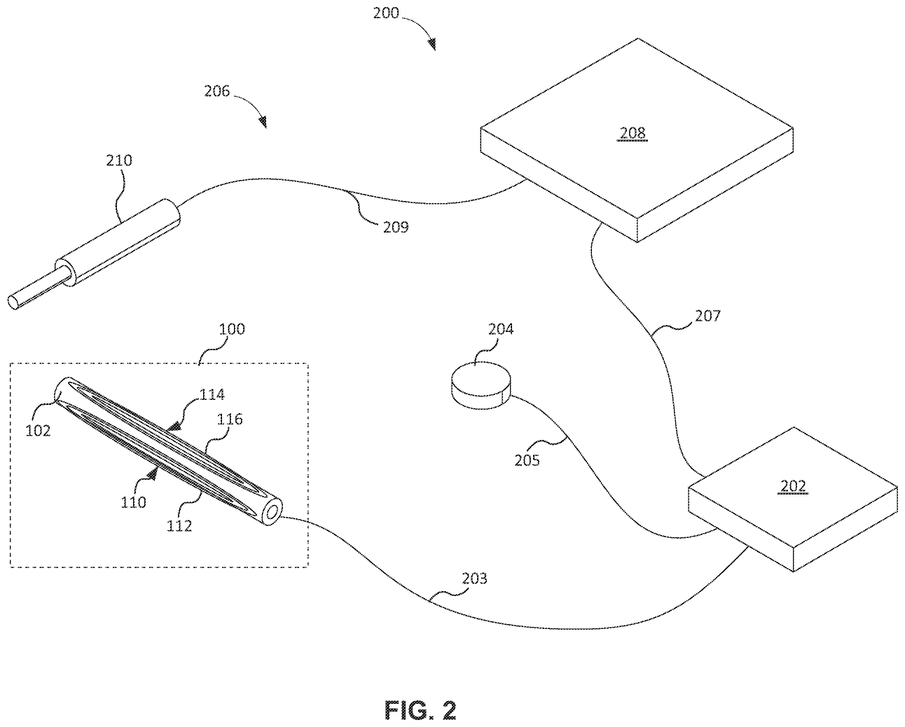

FIG. 2 shows an example system 200 including the stent apparatus 100 of FIG. 1. The system 200 in this embodiment comprises the apparatus 100, a control system 202, an override switch 204, and an electrosurgical device 206.

The electrosurgical device 206 may comprise a generator 208 and a surgical instrument 210. The generator 208 and surgical instrument 210 may be separate components, as shown in FIG. 2, or may be combined in a single device. The electrosurgical device 206 may be a monopolar or a bipolar electrosurgical device.

In this embodiment, the electrosurgical device 206 is an electrocautery device and the surgical instrument 210 is an electrocautery probe. Hereafter, the surgical instrument 210 will also be referred to as the probe 210. The generator 208 supplies power to the probe 210 to cauterize a target tissue during surgery. In other embodiments, the electrosurgical device 206 may be any other suitable electrosurgical device and the surgical instrument 210 may be any other electrified/powered surgical instrument.

In some embodiments, the electrosurgical device 206 is a conventional electrosurgical device. In other embodiments, the electrosurgical device 206 is customized to be operable at a standard voltage and amperage (for electrosurgery) as well as at a lower voltage and amperage to allow for low energy “mapping” of a bodily tissue as described in more detail below. In this embodiment, the electrosurgical device 206 is part of the system 200; however, in other embodiments, the electrosurgical device 206 may be commercial device which may be connected to the apparatus 100, control system 202, and override switch 204 to form the system 200.

When the probe 210 of the electrosurgical device 206 is powered and in proximity to the apparatus 100 (placed in a non-target tissue), the first and second coiled wires 112 and 116 of the first and second induction loops 110 and 114 may generate an electrical current via Faraday's Law in response to the electrical current and/or magnetic field of the probe 210.

The control system 202 is operatively connected to the apparatus 100. As used herein, “operatively connected” is intended to be inclusive of direct and indirect connections including wired and wireless connections. The control system 202 is operable to receive a signal (i.e., the electrical current) from the apparatus 100. In this embodiment, the control system 202 is connected to the apparatus 100 by at least one wire 203. The wire 203 is electrically connected to the first and second coiled wires 112 and 116. In some embodiments, the first and second coiled wires 112 and 116 are formed from the wire 203 such that the first and second coiled wires 112 and 116 and the wire 203 are substantially continuous. In other embodiments, the apparatus 100 may be connected to the control system 202 wirelessly.

The control system 202 may also be operatively connected to the electrosurgical device 206. The control system 202 may be operable to transmit a control signal to the generator 208. In some embodiments, the control system 202 may also be operable to receive data from the generator 208 including, for example, the current voltage and amperage being supplied to the probe 210. In this embodiment, the control system 202 is electrically connected to the generator 208 of the electrosurgical device 206 by a wire 207. In other embodiments, the control system 202 may communicate with the generator 208 wirelessly. In other embodiments, the generator 208 may be integrated with the control system 202 such that the control system 202 directly controls the power to the probe 210.

Optionally, the control system 202 may also be operatively connected to an override switch 204. In this embodiment, the override switch 204 is a foot switch and is connected to the control system 202 by a wire 205. The override switch 204 may be operable to prevent the control system 202 from sending a signal to the generator 208 to shut off power to the probe 210.

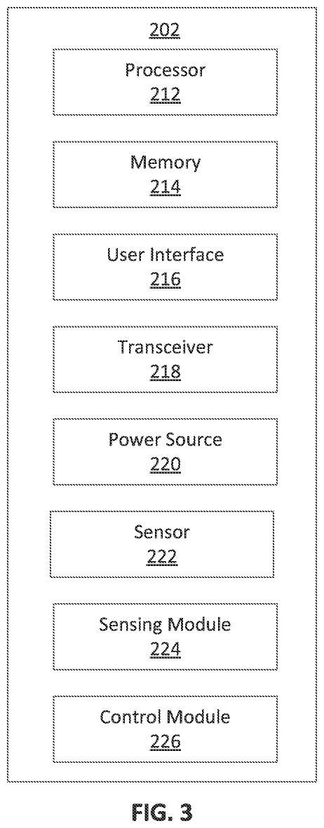

FIG. 3 is a block diagram of the control system 202. The control system 202 in this embodiment comprises a processor 212, a memory 214, a user interface 216, a transceiver 218, a power source 220, a sensor 222, at least one sensing module 224, and a control module 226.

The memory 214 is operatively connected to the processor 212. The memory 214 may store processor-executable instructions therein that, when executed, cause the processor 212 to implement one or more methods described herein.

The user interface 216 is operatively connected to the processor 212. The processor 212 may be configured to receive input from a user via the user interface 216 and/or display output to the user via the user interface 216.

The user interface 216 may comprise at least one input component and/or at least one output component. The input component may comprise, for example, at least one of a touchscreen, a keyboard, a keypad, a mouse, a microphone, or any other suitable type of input device. The output component may comprise, for example, at least one of a display screen, an audio output device (e.g., one or more speakers), a visual output device (e.g., one or more lights), or any other suitable type of output device.

The transceiver 218 may be configured to send and receive communications over a communication network such as the Internet. The communication network may comprise a wired or a wireless network. In some embodiments, the transceiver 218 comprises both a transmitter and a receiver sharing common circuitry. In other embodiments, the transceiver 218 comprises a separate transmitter and receiver.

The power source 220 may be configured to supply power to the control system 202. The power source 220 may be an internal power source (e.g., a battery) or an external power source. The external power source may be connected to the control system 202 by a wire or any other suitable means.

The sensor 222 may be configured to sense the signal from the apparatus 100 when the probe 210 approaches the apparatus 100. As used herein, “sensing” refers to detecting, measuring, or otherwise acquiring data or information related to the signal. In this embodiment, the signal is an electrical current and the sensor 222 comprises a galvanometer or ammeter that detects and measures the electrical current. In other embodiments, the sensor 222 comprises any other suitable sensing device. In some embodiments, the sensor 222 is capable of sensing fluctuations or variations of the electrical current generated from the apparatus 100 as the probe 210 is moved to different positions with respect to the apparatus 100.

In this embodiment, the sensor 222 is part of the control system 202. In other embodiments, the sensor 222 may be a separate component operatively connected to the control system 202 and operable to transmit sensed data to the control system 202 via the transceiver 218. In alternative embodiments, more than one sensor 222 may be provided to sense the signal from the apparatus 100.

At least one sensing module 224 may be configured to receive and process the signal from the sensor 222. The sensing module(s) 224 may process the signal data to determine a distance between the apparatus 100 and the probe 210, for example, based on the distinct current wave patterns received from the apparatus 100 via the sensor 222. In some embodiments, the data comprises sensed variations of the electrical current from the apparatus 100 as the probe 210 is moved to different positions with respect to the apparatus 100. The sensing module(s) 224 may process the sensed variations to “map” the bodily tissue in which the apparatus 100 is placed (e.g., the ureter or rectum), as described in more detail below.

Although only one sensing module 224 is shown in FIG. 3, it will be understood that the control system 202 may comprise more than one sensing module 224. In some embodiments, the control system 202 comprises three or more separate sensing modules 224. For example, the control system 202 may comprise one or more sensing modules 224 to receive data from the sensor 222 and one or more sensing modules 224 to receive data from the optional temperature and pressure sensors of the apparatus 100, if used.

In some embodiments, the sensing module(s) 224 determines a threshold value for the electrical current received from the apparatus 100. The threshold may be based on the minimum distance between the probe 210 and the apparatus 100 to avoid damaging the non-target tissue in which the apparatus 100 is placed. In some embodiments, the sensing module(s) 224 determines an integrated “safety” threshold based on one or more of a safe distance, temperature, time/duration, voltage and amperage of the probe 210 to avoid such damage. The threshold may be determined through empirical analysis and applying the Arrhenius equation to best fit the results. The threshold value(s) may be communicated to the control module 226 directly or may be displayed to the user via the user interface 216 (or both).

The control module 226 may receive processed data from the sensing module(s) 224 and/or user input via the user interface 216. The control module 226 may also receive user input via the override switch 204. The control module 226 may cause a control signal to be transmitted to the electrosurgical device 206 in response to data and/or user input indicating that the probe 210 is approaching the apparatus 100.

In some embodiments, the control module 226 has a pre-determined threshold value for the electrical current received from the apparatus 100 via the sensor 222 such that the control signal is transmitted once the threshold has been reached. In some embodiments, the control module 226 has a pre-determined integrated “safety” threshold as described above. The pre-determined threshold value(s) may be determined by the sensing module(s) 224 or may be pre-programmed into the control module 226 based on previous calculations.

The control signal may be transmitted to the generator 208 to shut off (or turn on) power to the probe 210. In some embodiments, the control signal activates or deactivates a switch (not shown), operably connected to the generator 208, to shut off/turn on power to the probe 210. Alternatively, or additionally, the control signal may be used by the generator 208 to increase or decrease the power to the probe 210 to a desired level without shutting off the probe 210 completely.

In other embodiments, the control module 226 displays instructions to the user via the user interface 216 to adjust the operation of the electrosurgical device 206 manually.

In some embodiments, the control module 226 generates a proximity indicator via the user interface 216 when a pre-determined threshold has been reached. As used herein, “proximity indicator” refers to a signal that indicates the proximity of the probe 210 to the apparatus 100. The threshold for generating the proximity indicator may be the same as the threshold for shutting off power to the probe 210 or may be a lower threshold. In some embodiments, the intensity of the proximity indicator varies based on variations in the signal as the probe 210 moves closer or farther away from the apparatus 100.

As one example, the proximity indicator may be an audible output signal such as a beep, tone, or alarm. In some embodiments, the pitch or volume of the audible output signal increases as the probe 210 gets closer to the apparatus 100 and the electrical current from the apparatus 100 increases. As another example, the proximity indicator may be a visual output signal such as a light or panel of lights. Optionally, the brightness of the visual output signal may increase, or the color may change, as the probe 210 gets closer to the apparatus 100.

Each of the sensing module(s) 224 and the control module 226 may be implemented as a processor (such as the processor 212) configured to perform the functions described above. Each module may be implemented as memory (such as the memory 214) containing instructions for execution by a processor (such as the processor 212), by hardware, or by a combination of instructions stored in memory and additional hardware, to name a few examples. The memory 214 may be internal or external to the processor 212.

In some embodiments, the control system 202 is in communication with one or more remote devices via the communication network. The remote device may comprise, for example, a client computer or server. In some embodiments, the remote device may comprise a mobile communications device such as a smart phone or tablet.

In some embodiments, the control system 202 may receive data from the sensor 222 (and optional heat and pressure sensors) and transmit the data to the remote device for processing. For example, the remote device may process data received from the control system 202 to generate an image of the non-target tissue based on the signal generated from the apparatus 100.

In some embodiments, the control module 226 is operable to receive input from the remote device. In other embodiments, the control module 226 may be omitted from the control system 202 and the remote device may send a control signal directly to the electrosurgical device 206 and/or display appropriate instructions to the user.

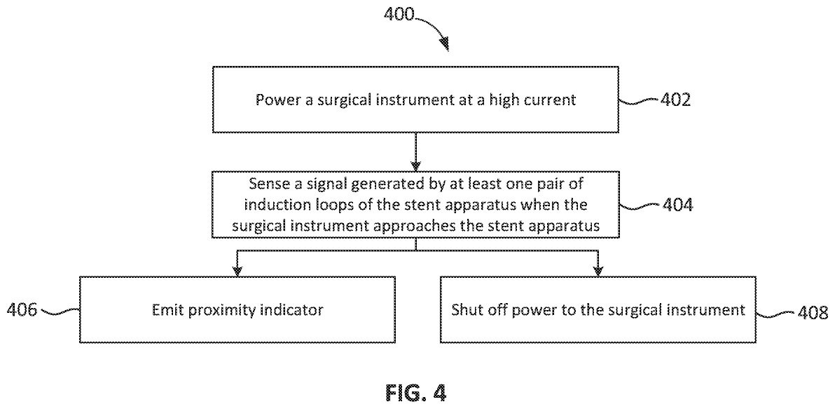

FIG. 4 is a flowchart of an example method 400, according to some embodiments. The method 400 may be implemented at the control system 202 of the system 200, operatively connected to the surgical instrument (i.e., probe) 210 and the stent apparatus 100.

Prior to the steps of the method 400, the apparatus 100 is placed in the desired non-target tissue during a surgical procedure. In some embodiments, the apparatus 100 is placed in the non-target tissue cytoscopically at the start of the operation. In some embodiments, the non-target tissue is the ureter, and the surgical procedure is a pelvic, gynecological, obstetrical, or colorectal surgery. In other embodiments, the non-target tissue may be the patient's rectum and the patient may be undergoing abdominal or pelvic surgery. In other embodiments, the non-target tissue may be any other suitable non-target tissue during any other surgical procedure and embodiments are not limited to only the ureter and rectum.

At block 402, the surgical instrument (probe) 210 is powered. The probe 210 is powered via the generator 208, which may be operated manually by the user or automatically via the control system 202. In this example, the probe 210 is powered at a high current (i.e., a high voltage and amperage) suitable for conducting electrosurgery. In some embodiments, the operating range for the probe 210 is between about 30 and about 80 watts of power. For example, the current may be at a level suitable for cauterizing, cutting, coagulating, desiccating, fulgurating, or otherwise altering the target tissue of the surgery.

At block 404, a signal is sensed, via the sensor 222, when the probe 210 approaches the apparatus 100. In this example, the signal is an electrical current generated by the first induction loop 110 and the second induction loop 114 by Faraday's Law in response to the electrical current and/or magnetic field of the probe 210.

Although block 404 is shown after block 402 in FIG. 4, it will be understood that the steps at blocks 402 and 404 may be performed substantially concurrently such that the surgical instrument 210 is powered as the signal generated by the apparatus 100 is sensed via the sensor 222.

At block 406, a proximity indicator (e.g., an audible or visual output signal) is generated by the user interface 216 as a warning to the user that the probe 210 is approaching the apparatus 100. The user may then move the probe 210 farther away from the non-target tissue in response to the audible or visual warning before any damage to the non-target tissue can occur.

At block 408, power is shut off to the probe 210 when the signal reaches a pre-determined threshold. The steps at block 408 may occur as an alternative to the steps at block 406 or may occur after block 406 if the user continues to move the probe 210 towards the apparatus 100.

In some embodiments, power to the probe 210 is automatically shut off via a control signal sent to the generator 208 by the control system 202. In these embodiments, the power may be shut off almost instantaneously or within a few seconds of the threshold being reached. In other embodiments, the control system 202 may display instructions to the user, via the user interface 216, indicating to the user to shut off the power manually. Thus, by shutting off power to the probe 210 as the probe 210 approaches the apparatus 100, damage to the non-target tissue can be avoided.

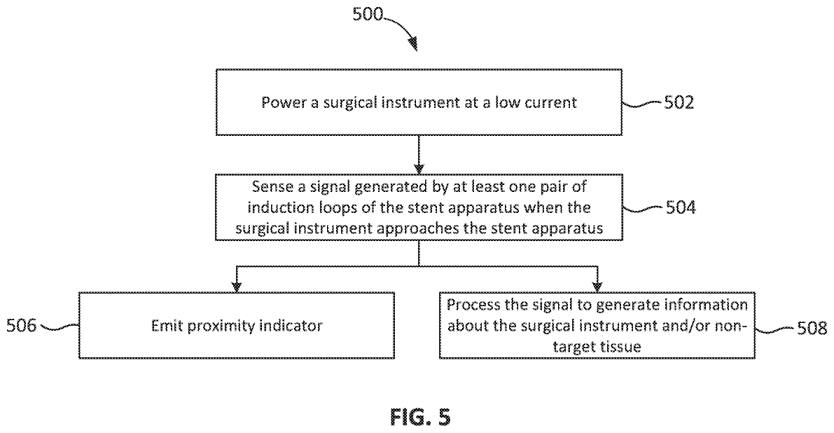

FIG. 5 is a flowchart of an example method 500, according to some embodiments. The method 500 may be implemented at the control system 202 of the system 200, operatively connected to the surgical instrument (i.e., probe) 210 and the stent apparatus 100.

Prior to the steps of the method 500, the apparatus 100 may be placed in a desired non-target tissue as discussed above with respect to the method 400.

At block 502, the surgical instrument (probe) 210 is powered at a low current (i.e., low voltage and amperage). In some embodiments, the probe 210 is powered about 10 watts of power or less. The low current may be atraumatic to tissue, particularly to the non-target tissue. As used herein, “atraumatic” or “non-damaging” refers to a current that does not significantly damage or otherwise alter a tissue. The steps at block 502 may otherwise be similar to the steps at block 402 of the method 400 described above.

At block 504, a signal is sensed, via the sensor 222, when the surgical instrument (probe) 210 approaches the apparatus 100. In this example, the signal is the electrical current generated from the first and second induction loops 110 and 114 of the apparatus 100.

Although block 504 is shown after block 502 in FIG. 5, it will be understood that the steps at blocks 502 and 504 may be performed substantially concurrently such that the surgical instrument 210 is powered as the signal generated by the apparatus 100 is sensed via the sensor 222.

At block 506, a proximity indicator is emitted via the user interface 216. The proximity indicator may comprise an audible and/or visual output signal. In some embodiments, the intensity of the audible or visual output signal varies as the probe 210 moves closer to, or farther from, the apparatus 100. For example, the pitch or volume of the audible output signal, or the brightness or color of the visual output signal, may vary based on the distance between the probe 210 and the apparatus 100.

In some embodiments, the probe 210 is powered at block 502 for a short “test” period and the proximity indicator is emitted in response to the signal generated by the apparatus 100 in order to locate the apparatus 100. The test period may thereby be used to confirm that it is safe to proceed with the electrosurgery.

A short test period may also be used to confirm the integrity of the Faraday circuit formed by the first and second induction loops 110 and 114 of the apparatus 100. A decrease or loss of the electrical current from the apparatus 100 may be used to indicate damage to the apparatus 100. Damage to the apparatus 100 may be indicative of a crush injury to the non-target tissue. In addition, confirming the integrity of the apparatus 100 may help to avoid false negative readings during the surgery.

In other embodiments, the probe 210 may be powered for a longer period of time and the resulting proximity indicator may be used to map the non-target tissue before proceeding with the surgery. In some embodiments, variations in the electrical current may be sensed by the sensor 222 as the probe 210 is moved to various positions with respect to the apparatus 100, causing variations in the emission or intensity of the proximity indicator. Mapping the non-target tissue may help the user better avoid the non-target tissue during the surgery.

At block 508, the signal data from the sensor 222 is processed to generate information about the probe 210 and/or the non-target tissue. The steps at block 508 may be an alternative to the steps at block 506 or in addition thereto. The data from the sensor 222 may be processed by the processor of the control system 202 or the data may be transmitted to a remote device to be processed.

In some embodiments, the sensor data is processed to generate an image of the non-target tissue. The image may be displayed to the user via the user interface 216 or via a user interface of the remote device.

In some embodiments, the data from the sensor 222 is processed to determine a distance between the apparatus 100 and the probe 210. For example, the distinct current wave form patterns may be used to determine the distance between the apparatus 100 and the probe 210 at a given time. In some embodiments, the data may be used to determine a suitable distance threshold for the probe 210 or an integrated “safety” threshold. The determined distance or threshold(s) may be saved in the memory 214 of the control system 202 and/or displayed to the user via the user interface 216. The threshold(s) may be used at block 406 of the method 400 as described above.

In some embodiments, data from the optional temperature and pressure sensors is also processed by the control system 202. This data may be used to calculate the integrated safety threshold. An increase in temperature may also be used to indicate that the probe 210 is too close to the apparatus 100 or that the probe 210 has been in the proximity of the apparatus 100 for too long of a time period. An increase in pressure may indicate that the apparatus 100, and thus the non-target tissue, is being crushed and/or damaged.

Once the apparatus 100 has been located and/or the non-target tissue has been mapped, the current to the probe 210 can be increased to a suitable level to conduct the electrosurgery.

In some embodiments, during the surgical procedure, the system 200 may be used to continue to monitor the integrity of the apparatus 100. For example, the power to the probe 210 may periodically be lowered back to the low, atraumatic current level and the probe 210 may be brought into proximity of the apparatus 100 to generate an electrical current therefrom. A decrease or loss of the current may indicate that the apparatus 100 is damaged and that the non-target tissue potentially incurred a crush injury.

Thus, embodiments of the apparatuses, systems, and methods disclosed herein may be used to reduce or prevent injuries to non-target tissues during electrosurgery, thereby reducing patient morbidity and avoiding the need for additional costly surgeries to repair such injuries. The apparatuses and systems are also relatively inexpensive and easy to use and can be incorporated into a variety of surgical procedures with a variety of surgical instruments.

Although particular embodiments have been shown and described, it will be appreciated by those skilled in the art that various changes and modifications might be made without departing from the scope of the disclosure. The terms and expressions used in the preceding specification have been used herein as terms of description and not of limitation, and there is no intention in the use of such terms and expressions of excluding equivalents of the features shown and described or portions thereof. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1. A stent apparatus comprising:
a tubular body to be positioned in a bodily tissue;
at least one pair of induction loops in or on the tubular body, the at least one pair of induction loops comprising a first induction loop and a second induction loop, the first induction loop substantially orthogonal to the second induction loop; and
wherein the at least one pair of induction loops is capable of generating an electrical current in the presence of one or more of magnetic, electrical, and radiofrequency energy.

2. The stent apparatus of claim 1, wherein each induction loop of the at least one pair of induction loops comprises a respective coiled wire.

3. The stent apparatus of claim 1, wherein each induction loop of the at least one pair of induction loops comprises conductive ink printed on the tubular body.

4. The stent apparatus of claim 1, wherein the at least one pair of induction loops is disposed on an outer surface of the tubular body.

5. The stent apparatus of claim 1, wherein the at least one pair of induction loops is embedded in the tubular body.

6. The stent apparatus of claim 1, wherein the at least one pair of induction loops further comprises a third induction loop and a fourth induction loop, the third induction loop substantially orthogonal to the fourth induction loop.

7. The stent apparatus of claim 1, further comprising at least one of a pressure sensor, a temperature sensor, and a light source.

8. A system for sensing a surgical instrument in a bodily tissue, comprising:
a stent apparatus to be positioned in the bodily tissue, the stent apparatus comprising at least one pair of induction loops that generate a signal in the presence of the surgical instrument; and
a control system operatively connected to the stent apparatus, the control system comprising a sensor operable to receive the signal from the at least one pair of induction loops.

9. The system of claim 8, wherein the signal is an electrical current and the sensor comprises a galvanometer or an ammeter.

10. The system of claim 8, wherein the control system further comprises a user interface that generates a proximity indicator in response to the signal from the stent apparatus.

11. The system of claim 10, wherein the proximity indicator is an audible or visual output signal.

12. The system of claim 8, wherein the control system is operatively connected to a generator that supplies power to the surgical instrument, and wherein the control system is operable to transmit a control signal to the generator to shut off power to the surgical instrument in response to the signal from the stent apparatus.

13. A method at a control system comprising a sensor, the control system operatively connected to a surgical instrument and a stent apparatus comprising at least one pair of induction loops, the method comprising:
powering the surgical instrument; and
sensing a signal, via the sensor, generated from the at least one pair of induction loops when the surgical instrument approaches the stent apparatus.

14. The method of claim 13, further comprising shutting off power to the surgical instrument in response to the signal.

15. The method of claim 14, wherein power is shut off when the signal reaches a pre-determined threshold, the pre-determined threshold being based on one or more of a safe distance, temperature, time/duration, voltage and amperage of the surgical instrument.

16. The method of claim 13, wherein powering the surgical instrument comprises supplying a low current to the surgical instrument, the low current being atraumatic to tissue.

17. The method of claim 13, further comprising generating a proximity indicator in response to the signal, the proximity indicator comprising an audible or visual output signal.

18. The method of claim 17, wherein the intensity of the audible or visual output signal varies in response to variations in the signal when the surgical instrument moves closer or farther away from the stent apparatus.

19. The method of claim 13, further comprising processing the signal to generate an image of a non-target bodily tissue.

20. The method of claim 13, further comprising sensing a decrease or loss of the signal to indicate damage to the stent apparatus.
